(19) United States
(12) Patent Application Publication
(10) Pub. No.: US 2008/0241844 A1
(43) Pub. Date: Oct. 2, 2008

(54) Devices and Methods for the Performance of Miniaturized In Vitro Assays

(75) Inventor: Gregory J. Kellogg, Cambridge, MA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: NETWORK BIOSYSTEMS, INC., Woburn, MA (US)

(21) Appl. No.: 12/027,252

(22) Filed: Feb. 6, 2008

Related U.S. Application Data
(60) Provisional application No. 60/888,407, filed on Feb. 6, 2007.

(51) Int. Cl. C12Q 1/68; C12M 1/34

(52) U.S. Cl. 435/6; 435/287.2

(57) ABSTRACT

This invention relates to methods and apparatus for performing microanalytic and microsynthetic analyses and procedures. The invention specifically provides devices and methods for performing miniaturized in vitro assays on biological samples, such as the polymerase chain reaction and Sanger sequencing reactions. Methods specific for the apparatus of the invention for performing PCR are provided.

### CROSS-REFERENCE TO RELATED APPLICATION

This application claims the benefit of the filing date, under 35 U.S.C. §119(e), of U.S. Provisional Application Ser. No. 60/888,407, filed Feb. 6, 2007, which is hereby incorporated by reference in its entirety.

### BACKGROUND OF THE INVENTION 1. Field of the Invention

This invention relates to methods and apparatus for performing microanalytic and microsynthetic analyses and procedures. In particular, the invention relates to microminiaturization of genetic, biochemical and bioanalytic processes. Specifically, the present invention provides devices and methods for the performance of integrated and miniaturized nucleic acid assays, particularly amplification assays. These assays may be performed for a variety of purposes, including but not limited to forensics, life sciences research, and clinical and molecular diagnostics. The invention may be used on a variety of liquid samples of interest, including bacterial and cell cultures as well as whole blood, bodily fluids and processed tissues, and nucleic acids at various conditions of purity. Methods for performing any of a wide variety of such microanalytical or microsynthetic processes using the apparatus of the invention are also provided.

2. Background of the Related Art

Recent developments in a variety of investigational and research fields have created a need for improved methods and apparatus for performing analytical, particularly bioanalytical assays at microscale (i.e., in volumes of less than 100 μL). The primary developmental approach has been and will continue to be to miniaturize existing assays in order to decrease compound and reagent costs (that scale with the volume required for performing the assay). Miniaturization has been accompanied by the development of more sensitive detection schemes, including both better detectors for conventional signals (e.g., calorimetric absorption, fluorescence, and chemiluminescence) as well as new chemistries or assay formats (e.g., imaging, optical scanning, and confocal microscopy).

Miniaturization can also confer performance advantages. At short length scales, diffusionally-limited mixing is rapid and can be exploited to create sensitive assays (Brody et al., 1996, Biophysical J. 71: 3430-3431). Because fluid flow in miniaturized pressure-driven systems is laminar, rather than turbulent, processes such as washing and fluid replacement are well-controlled. Microfabricated systems also enable assays that rely on a large surface area to volume ratio such as those that require binding to a surface and a variety of chromatographic approaches.

In the biological and biochemical arts, analytical procedures frequently require incubation of biological samples and reaction mixtures at temperatures greater than ambient temperature. Moreover, many bioanalytical and biosynthetic techniques require incubation at more than one temperature, either sequentially or over the course of a reaction scheme or protocol.

One example of such a bioanalytical reaction is the polymerase chain reaction. The polymerase chain reaction (PCR) is a technique that permits amplification and detection of nucleic acid sequences. See U.S. Pat. No. 4,683,195 to Mullis et al. and U.S. Pat. No. 4,683,202 to Mullis. This technique has a wide variety of biological applications, including for example, DNA sequence analysis, probe generation, cloning of nucleic acid sequences, site-directed mutagenesis, detection of genetic mutations, diagnoses of viral infections, molecular “fingerprinting,” and the monitoring of contaminating microorganisms in biological fluids and other sources. The polymerase chain reaction comprises repeated rounds, or cycles, of target denaturation, primer annealing, and polymerase-mediated extension; the reaction process yields an exponential amplification of a specific target sequence.

A second example of a bioanalytical reaction utilizing thermal cycling is the Sanger sequencing reaction using either fluorescently-labeled primers or dye-terminators. In dye-terminator Sanger sequencing, four distinct fluorescent molecules label terminators corresponding to each of the four nucleotides; a population of dye-labeled fragments is generated via thermal cycling. The fragments are then analyzed, conventionally, through electrophoresis using laser-induced fluorescence: Electrophoresis identifies a fragments size, while the specific fluorescence of the terminator determines the identity of the terminal base of the fragment. This is the basis for the majority of currently-available genomic sequencing technologies (Smith et al. 1986 “Fluorescence detection in automated DNA sequence analysis”. Nature. 321(6071):674-9).

Methods for miniaturizing and automating PCR are desirable in a wide variety of analytical contexts, particularly under conditions where a large multiplicity of samples must be analyzed simultaneously or when there is a small amount of sample to be analyzed. Miniaturization of PCR addresses both these concerns, since typically small amounts of sample can be used and a multiplicity of reaction can be performed on a single substrate such as a microchip.

In addition to PCR, other in vitro biochemical and bioanalytic processes , include, but are not limited to, ligase chain reaction as disclosed in U.S. Pat. 4,988,617 to Landegren and Hood, are known and advantageously used in the prior art. More generally, several important methods known in the biotechnology arts, such as nucleic acid hybridization and sequencing, are dependent upon changing the temperature of solutions containing sample molecules in a controlled fashion. Automation and miniaturization of the performance of these methods are desirable goals in the art.

Mechanical and automated fluid handling systems and instruments produced to perform automated PCR, particularly miniaturized to microscale (0.5-100 μL) have been disclosed in the prior art.

U.S. Pat. No. 5,304,487, issued Apr. 19, 1994 to Wilding et al. teaches fluid handling on microscale analytical devices.

International Application, Publication No. W093/22053, published 11 Nov. 1993 to University of Pennsylvania disclose microfabricated detection structures.

International Application, Publication No. W093/22058, published 11 Nov. 1993 to University of Pennsylvania disclose microfabricated structures for performing polynucleotide amplification.

Wilding et al., 1994, Clin. Chem. 40: 43-47 disclose manipulation of fluids on straight channels micromachined into silicon.

Kopp et al., 1998, Science 280: 1046 discloses microchips for performing in vitro amplification reactions using alternating regions of different temperature.

Valveless microfluidics apparatus, in which surface forces, microscale structure, and applied pressure are used to gate fluids in microfluidic devices, has been shown to be applicable to a wide range of fluid types, from reagents in buffer solution; to biological fluids such as blood and low surface-tension fluids such as solutions containing surfactant; organic solvents and oils, and inorganic oils such as silicone oil. See, for example, U.S. Pat. Nos. 6,143,248, 6,706,519, 6,953,550, and 7,020,355. The use of valveless microfluidics greatly reduces the cost and complexity of microfluidic devices by allowing their fabrication using high throughput processes such as injection molding, surface treatment, and bonding. One shortcoming of such devices as conventionally used, however, is that fluids undergoing incubations or high-temperature processes such as PCR are typically lost from the device or from the specific chamber in which they are being held due to a combination of evaporative loss and flow from creation of bubbles in the fluid. Bubbles in the reaction mixture also impede detection of reaction products and analytes when the reaction mixture is interrogated, inter alia, spectrophotometrically.

Thus, there exists a need in the art for devices and methods that permit miniaturization of temperature-dependent assays, particularly assays involving incubating a reaction mixture at temperatures greater than ambient, under conditions where loss of reaction mixture volume and creating of vapor-containing bubbles are minimized.

### SUMMARY OF THE INVENTION

The invention provides apparatus and methods for performing microscale processes on a solid substrate, preferably a fabricated microfluidics microchip, wherein the microfluidics components are arranged and the methods performed to minimize evaporative and convective loss of reaction mixture volumes and to minimize creation, size or both of vapor-containing bubbles in the reaction mixture. In preferred embodiments, fluidic movement on the substrate is provided by externally-applied pressure. In yet further preferred embodiments, pressure greater than ambient pressure is applied to the reaction mixture to minimize evaporative and convective loss of reaction mixture volumes and to minimize creation, size or both of vapor-containing bubbles in the reaction mixture. The pressure is generally provided by a pressurization gas (e.g., purified nitrogen, air, argon, and mixtures there) which forms at least one liquid-gas interface between the pressurization gas and the reaction mixture.

The microchip apparatus of the invention is provided to perform miniaturized biological assays, particularly nucleic acid amplification and nucleic acid detection assays. A first element of the apparatus of the invention is a microchip substrate comprising fluid (sample) inlet ports, fluidic microchannels, reagent reservoirs, collection chambers, detection chambers and sample outlet ports, generically termed “microfluidic structures”. Microchip substrates of the invention also preferably comprise air outlet ports and air displacement channels. The air outlet ports and in particular the air displacement ports provide a means for fluids to displace air, thus ensuring uninhibited movement of fluids in the microfluidics structures on the chip. The microchip substrate is adapted for heating at particular positions on the substrate that comprise reaction chambers; in certain embodiments heating is performed by an external heating apparatus, while in alternative embodiments the microchip contains heating elements for raising the temperature of fluids contained in said reaction chambers to temperatures greater than ambient temperatures. Specific sites on the microchip also preferably comprise elements that allow fluids or the components thereof to be analyzed.

The microchip substrates of the invention are provided comprising microfluidic structures that perform biological assays such as nucleic acid amplification assays and permit the products of such assays to be detected, as described in further detail below. These microchip substrates are illustrated for clarity with regard to a single embodiment. However, microchip substrates comprising a multiplicity of such microfluidic structures for performing biological assays such as nucleic acid amplification assays and permit the products of such assays to be detected are provided by the invention, wherein the microfluidics structures are arrayed on the surface of the microchip substrate with a density determined by the size of the microchip substrate and the volumetric capacity of the chambers and reservoirs comprising the microfluidic structures as disclosed herein.

In certain embodiments, the reaction chamber is fluidly connected to one or a plurality of microchannels having a length relative to the reaction chamber wherein heating of a reaction mixture in the presence of a pressurization gas and within the reaction chamber does not heat the liquid-gas interfaces of the reaction mixture with the pressurization gas to a temperature greater than 40° C. below the reaction mixture boiling point; preferably, the liquid-gas interfaces of the reaction mixture with the pressurization gas are not heated to a temperature greater than 20° C. below the reaction mixture boiling point. For example, in performing PCR comprising cyclic thermal treatments of the reaction mixture (e.g., when heating the reaction mixture to about 95° C.), the liquid gas interfaces do not reach a temperature greater than about 80° C.; preferably, the liquid gas interfaces do not reach a temperature greater than about 70° C.; more preferably, the liquid gas interfaces do not reach a temperature greater than about 60 C.

In general, the reaction chamber and a portion of each of the first and second microchannels in fluid communication therewith may be filled with a reaction mixture, as described herein. In such instances, a liquid-gas interface is formed between the reaction mixture and the pressurization gas within each of the first and second microchannels. In certain embodiments, heating of the reaction chamber does not heat the first and second microchannels at the liquid-gas interface between the reaction mixture and the pressurization gas to a temperature greater than 40° C. below denaturing temperature; preferably, wherein heating of the reaction chamber does not heat the first and second microchannels at the liquid-gas interface between the reaction mixture and the pressurization gas to a temperature greater than 20° C. below denaturing temperature.

The invention thus provides a microchip substrate having microfluidics structures as described herein for performing in vitro reactions. These include mixing of a biological sample with amplification reaction reagents, including deoxyribonuclotide triphosphates (dNTPs), dideoxyribonucleotide triphosphates (ddNTPs), dye-labeled deoxyribosenuclotides, dye-labeled dideoxyribosenucleotides, polymerase enzyme, primers, dye-labeled primers, and appropriate salts, buffers and additives; and thermal cycling to effect the in vitro reaction, as well as analysis of the resulting product. The dye labels may be independently selected from the dichroic, radioactive, and fluorescent dyes familiar to those skilled in the art. In preferred embodiments, the in vitro reaction is an amplification reaction. In one embodiment, the amplification reaction is PCR. In alternative preferred embodiments, the in vitro reaction is a Sanger sequencing reaction.

In certain preferred embodiments, the microchip substrates of the invention are provided with a multiplicity of microfluidics structures that enable to microchip to process several samples simultaneously. In these embodiments, multiple copies of an arrangement of microfluidics structures for performing in vitro reactions are arrayed on the substrate, and sample input ports or reservoirs provided for each copy, thereby permitting processing of multiple samples. Each process performed on such substrates may be identical or different

In addition, when performing an amplification reaction, the portion of a sample DNA can be independently amplified, by the choice of amplification primers provided in each of the individual copies of the microfluidics structures arrayed on the microchip, thereby permitting amplification “multiplexing” of a particular sample. Alternatively, the same primers can be provided to process in parallel multiple samples for amplification of the same target fragment in the DNA of each sample. Independent thermal cycling profiles, including the temperature used for each step of the amplification cycle, temperature ramp-rates, and hold times, may be individually programmed into the instrument for each of the microfluidics structures or for each of the samples processed.

The invention advantageously permits simultaneous, independent thermal cycling of a multiplicity of different samples, independent analysis (e.g., amplification) of different target fragments from a particular sample, or both. Since particular copies of the microfluidics structures can be arranged in microfluidic isolation from other copies on the microchip substrate, portions comprising less than all of the microfluidics structures can be discretely used and the remainder retained for future use.

Additional microfluidics components useful in the microchip substrate include metering structures used to distribute aliquots of reagent to each of a multiplicity of mixing structures, each mixing structure being fluidly connected to one of a multiplicity of sample reservoirs, thereby permitting parallel processing and mixing of the samples with a common reagent. This reduces the need for automated reagent distribution mechanisms, reduces the amount of time required for reagent dispensing (that can be performed in parallel with distribution of reagent to a multiplicity of reaction chambers), and permits delivery of small (nL-to-μL) volumes without using externally-applied electromotive means.

The assembly of a multiplicity of collection chambers on the microchip substrates of the invention also permits simplified detectors to be used, whereby each individual collection/detection chamber can be analyzed by methods familiar to those skilled in the art. Finally, the microchip substrates of the invention are advantageously provided with sample and reagent entry ports for filling with samples and reagents, respectively, that can be adapted to liquid delivery means known in the art (such as micropipettors).

It is an advantage of the microchip substrates of the present invention that the fluid-containing components are constructed to contain a small volume, thus reducing reagent costs, reaction times and the amount of biological material required to perform an assay. It is also an advantage that the fluid-containing components are sealed, thus eliminating experimental error due to differential evaporation of different fluids and the resulting changes in reagent concentration. Because the microfluidic devices of the invention are completely enclosed, both evaporation and optical distortion are reduced to negligible levels. It is an additional advantage of the microchip substrates as provided herein that reactions can be performed under greater-than-atmospheric pressure. It is a further advantage of the microchip substrates of the present invention that the sealing may be accomplished without the use of physical valves or the addition of capping oils, which greatly simplifies their operation and purification of any products produced therein.

The microchip substrates of the invention also advantageously permit “passive” mixing and valving, i.e., mixing and valving are performed as a consequence of the structural arrangements of the components on the microchip substrates (such as shape, length, position on the microchip substrate surface, and surface properties of the interior surfaces of the components, such as wettability as discussed below), and the dynamics of the applied external pressure, and permit control of assay timing and reagent delivery.

Certain preferred embodiments of the apparatus of the invention are described in greater detail in the following sections of this application and in the drawings.

### BRIEF DESCRIPTION OF THE DRAWINGS

FIG. 1 is a schematic diagram of a 2-sample thermal cycling chip (e.g., 0.5 mm deep×1 mm wide channels) according to the invention for placement on a thermal cycler top. The ports at the left allow sample to be added or removed and are pressurized during cycling

FIG. 2 illustrates the progress of bubble formation in a reaction chamber with (right) and without (left) added pressure.

FIG. 3 is a graph showing the number of basepairs called with various Phred QV scores for Sanger sequencing reaction products obtained by thermally cycling the chip of FIG. 1 (Chip) compared to those thermally cycled in tubes (Tube).

FIG. 4 is a graphic illustration of a Peltier device with a chip according to the invention.

FIG. 5 is a graph illustrating the temperature measured at the reaction chamber (sample), chip top, and Peltier surface upon thermal cyclic of the chip and Peltier device of FIG. 4.

FIG. 6 is a photograph of a gel illustrating a 1.8 kb product retrieved from PCR of a 3 μL sample from a chip of the construction shown in FIG. 4.

FIG. 7 is an illustration of how multiple reaction chambers (e.g., four) can be tiled along a single heating and cooling surface (bottom).

FIG. 8 illustrates a chip of the invention comprising an insulating air pocket that can be used to further reduce the temperature at the liquid/vapor interface of the sample.

FIG. 9 is a graph illustrating that the insulating air pocket in the chip of FIG. 8 reduces the temperature at the top surface of the chip.

### DETAILED DESCRIPTION OF PREFERRED EMBODIMENTS

This invention provides a microchip substrate for performing in vitro microanalytical and microsynthetic assays of biological samples, particularly amplification reactions. The invention provides said microchip substrates adapted for performing in vitro reactions, such as the polymerase chain reaction (PCR) under condition of elevated pressure (greater than atmospheric pressures). The microchip substrates of the invention, and methods for using said substrates, are specifically adapted for performing said assays

This invention provides microchip substrates wherein the microfluidic components are arrayed on and in the substrate to minimize evaporative and convective losses of a reaction mixture, particularly a PCR reaction mixture, from the substrate. The invention relies on various combinations of applied pressure, the incorporation of long, narrow channels at the inlet and outlet of the chamber to be held at high temperature, differential heating of those channels and the chamber, and surface properties (including both material choice and surface treatments) to prevent condensation of vapor.

Applied pressure is used to prevent or diminish the formation of bubbles due to heating of the liquid and is generated by the introduction of a pressurized gas into the microchip structures of the invention. The applied pressure provides at least one, and generally two liquid-gas interfaces between the pressurized gas the liquid being heated, thereby confining the liquid under increased pressure without the use of physical valves. The temperature at which bubbles nucleate as well as the boiling point of liquids are elevated at pressures above ambient. This can be seen from the empirical equation for vapor pressure as a function of temperature (http://hyperphysics.phy-astr.gsu.edu/hbase/kinetic/vappre.html#c6),

Pᵥ=2427.9-60.726T+0.44048T²

where Pᵥ is in mmHg, and T is in degrees C. When the vapor pressure Pᵥ equals that of the gas above the water, it boils, and thus when the pressure above a liquid is increased, the boiling point temperature increases. For example, a saturated vapor pressure of 3 atmospheres (2180 mmHg) results in a boiling point elevation to 135° C. while 2 atmospheres results in 125° C. While this expression is accurate only near 100° C., the general conclusion is valid; increases in applied pressure increase the boiling point.

Application of moderate pressure thus increases the boiling point and reduces the likelihood that the temperatures of a given processes will approach the boiling point, thus diminishing or eliminating the number of bubbles formed. An additional advantage of applied pressure is that it reduces the possibility that dissolved oxygen will form bubbles: (http://www.sra.dst.tx.us/srwmp/mr_water_wizard/default.asp?page=faq&group=5#faq). In addition to reducing the possibility that bubbles will form locally on the heated surface, applied pressure reduces the size of the resulting bubbles relative to that at ambient pressure: Bubbles at elevated pressures containing the same number of vapor molecules as those at one atmosphere are smaller by the ratio of the pressures. Both effects—decreasing the number of water molecules that vaporize within bubbles as well as compressing the resulting bubbles—greatly decrease the total volume of the bubbles which displace the liquid.

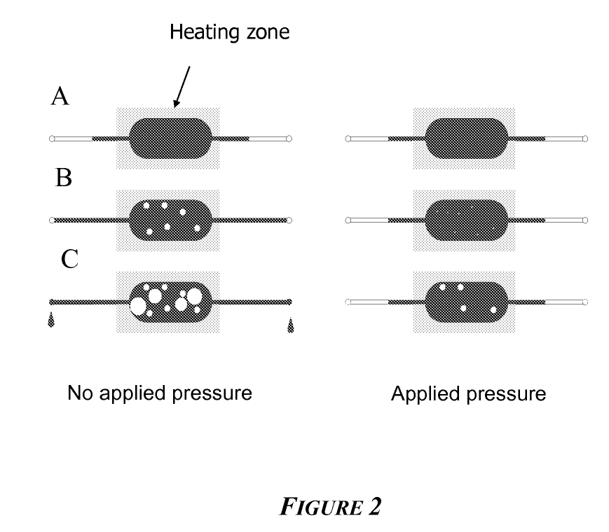

FIG. 2 illustrates the effect of applied pressure on the rate of bubble formation within a microfluidic reaction chamber. In the left panel, a chamber with channels open to the atmosphere is heated to 95° C. during PCR by the use of a heater situated against one wall of the chamber thin enough to allow heat transfer sufficient for temperature changes between 1° C. and 20° C./second (A). As heat is applied, the local temperature on the wall closest to the heater may rise above the boiling point, especially in cycling processes such as PCR, and bubbles form along the surface, nucleating on the microstructure of the surface (B). These bubbles grow as liquid becomes vapor and drive liquid toward the ports, eventually leading it to emerge (C). In the right panel, a chamber at an elevated pressure undergoes similar heating (A). The resulting bubbles are far smaller (B, C). Additionally, as the temperature is lowered during PCR cycling bubbles are seen to shrink significantly as vapor recondenses into the liquid. A second source of bubbles is outgassing due to reduced solubility of atmospheric gasses as temperature increases. Application of pressure increases gas solubility at higher temperatures and prevents significant outgassing.

The temperature at which biological processes occur does not increase as steeply with applied pressure as does the boiling point. For example, PCR may be performed with a denaturing temperature of 94° C. even at applied pressures grater than 5000 atmospheres (see, for example, U.S. Pat. No. 6,753,169). As a result, the difference between denaturation temperature and boiling point of 6° C. at standard atmospheric conditions increases to 40° C. at three atmospheres and 30° C. at two atmospheres. At a temperature this far below the boiling point, the nucleation and growth of bubbles is far less and resulting fluid flow greatly decreased relative to the behavior at standard atmospheric conditions.

As used herein, it will be understood that atmospheric pressure at sea level is about 100 kPa. Elevated pressures will be at least 150 to 300 kPa, and can be 400- 600 kPa, or as much as 800 kPa to over 1 MPa, the limitations in pressure being a function of the structural integrity of the microchip substrate, the capacity of the external pump or other pressure source (e.g., gas cylinder) providing the pressure, and the strength of the seal (typically an O-ring seal) between ports on the microchip substrate and the pump.

A second aspect of the invention is the minimization of loss of water vapor from the exposed liquid interface. Even if bubbles do not form, small volumes can evaporate quickly due to the fact that there is a large ratio of exposed liquid/vapor interfacial area to the overall volume of the liquid. Evaporative loss in the absence of boiling or convection is due to three things: The vapor pressure of the liquid, which is in turn a function of the temperature of the liquid/vapor interface and the pressure; diffusion, the process by which vapor molecules are transported from the interface; and the surface area of the liquid/vapor interface(s).

This aspect of the invention is addressed in certain embodiments by differential heating. This is illustrated in FIG. 2, right panel. The chamber containing the bulk of the liquid is allowed to reach desired temperatures near the boiling point while under applied pressure, to reduce the formation of bubbles. The inlet and outlet arms are not heated. A small amount of liquid is retained in these channels of sufficient length that the temperature at the liquid/vapor interfaces is significantly lower than the boiling point, with a correspondingly low vapor pressure. If these surfaces are exposed to circulating room air and losses from them are convective—the greatest possible losses—the total evaporative loss per unit time is given by the Langmuir equation (http://van.physics.uiuc.edu/qa/listing.php?id=1440):

(mass loss rate)/(unit area)=(vapor pressure−ambient partial pressure)*sqrt((molecular weight)/(2*pi*R*T))

where the molecular weight of water is 0.018 kg/mole and the gas constant R=8.3124 J/mole−K and pressures are measured in Pascal (1 atmosphere=760 mmHg=1.01×10⁵ Pa). Since this expression gives the evaporation rate per area, it is clear that larger exposed liquid-vapor surfaces result in larger evaporation rates. Containing a heated liquid in a chamber such that the only liquid/vapor interface is within a small channel leading from the chamber will result in a far lower evaporation rate than that experienced by the equivalent volume in the form of a droplet.

Theoretical calculations and empirical evidence suggest that water vapor evaporation from unsealed reaction chambers on the microchip can be sufficiently significant that applying pressure as set forth herein improves performance of the reactions performed on the chip.

A third aspect of the invention relies on maintaining the liquid interface temperature below the boiling point or the maximum temperature of the bulk of the liquid. By maintaining a lower temperature, the vapor pressure is reduced, and evaporative loss is further reduced. The Langmuir equation above overestimates the evaporation rate because it assumes that all vapor is immediately removed from the vicinity of the liquid interface and does not recondense on the surface. Empirical observation shows that a 1 uL droplet evaporates at ≈0.1 uL/sec when placed on a hot plate at 95° C. The estimated evaporation for the same volume in which the only liquid/vapor interfaces exposed are those contained in two 100 um diameter channels is 2×10⁻³ uL/sec. If the liquid-vapor interfaces are held at 60° C., the decrease in vapor pressure leads to an estimate of 4.7×10⁻⁴ μL/sec.

In alternative embodiments (which it will be recognized could be used instead of or in combination with other embodiments set forth herein), the microfluidics components on the substrate are arranged to provide long, narrow, and unfilled channels leading away from the small liquid-vapor interfaces discussed above. As liquid evaporates from the interface, it is transported through diffusion in the air within the channel. It may diffuse back into the liquid, in which case it may recondense; it may strike a wall of the channel, in which case it may either condense or rebound; or it may diffuse down the channel toward the port. If means are employed to prevent condensation of liquid on the channel surface, the transport of vapor is described by the one-dimensional diffusion equation:

$$D ∇ \cdot ( ∇ c ) = \dot{c}$$

$$D \frac{{\partial }^{2} c}{\partial {x}^{2}} = \frac{\partial c}{\partial t}$$

where D is the diffusion constant of water vapor in air, c is the concentration of water vapor, x is the dimension along the channel and t is time. At steady-state, if x=0 is the liquid-vapor interface, one finds

$$c = {{c}_{0} ( 1 - x / l )}^{10}$$

$$Φ = - D \frac{\partial c}{\partial x} = \frac{{Dc}_{0}}{l}$$

where c₀ is the concentration of vapor at the liquid interface and l is the length of the channel and Φ is the flux of water vapor in the positive x direction (down the channel) in units of concentration/second. It is assumed that the distal end of the channel (x=1) has c=0, as for a port opening into an environment where vapor is rapidly carried away (e.g., stirred air).

The diffusion constant D of water vapor in air is 0.242 cm²/s. (http://home.att.net/numericana/answer/gas.htm). The initial concentration c₀ is given by the vapor pressure over the liquid surface. The saturated vapor density at 100° C./1 atm (760 mmHg)) is 598 g/m³. (http://hyperphysics.phy-astr.gsu.edu/hbase/kinetic/watvap.html#c1). For a channel of length 5 mm, this results in a flux of 0.000289 g/(cm² sec). If there are two square channels of cross-section 100 μm, the overall mass loss rate is 2*Φ*0.01 cm*0.01 cm=2.9×10⁻⁸ g/sec or 2.9×10⁻⁵ μL/sec. This corresponds to a loss of 0.1 μL over 1 hour. The loss during a 30 cycle thermal cycling process can be estimated as follows: the losses are dominated by the high temperature portion of the cycle, so that the total time at 95° C. may be used. At 95° C., the density of vapor is 499 g/m³. An extremely long, high-temperature process would be a Sanger cycling reaction of 50 cycles with 25 second holds at 95° C., for a total of 3000 seconds. The equation shows that the loss rate would be 2.42×10⁻⁵ μL/sec for the structure described above, or a total loss of 73 nL during the cycling. For an initial volume of 500 nL, this corresponds to an 15% loss, which is acceptable and within the normal range for small-volume thermal cycling reactions.

The above analysis relies on the inability of vapor to condense on the channel surfaces. This may be effected in two ways: through a different form of localized heating, in which the channels are maintained at a higher temperature than the reaction chamber; or the use of hydrophobic materials for constructing entrance and exit channels to suppress condensation. Hydrophobic materials may include the base material of the chip (e.g., polypropylene or poly(tetrafluoroethylene), i.e., Teflon®) or could be comprised of surface coatings physically deposited or chemically attached to the chip material. Furthermore, even in the absence of special surface treatments, vapor does not immediately condense as uniform film that grows through the deposition: Droplets form, typically at imperfections or inhomogeneities on the surface which are energetically favorable for condensation. It is these limited number of droplets that then grow as vapor condenses on their surface. Using the analysis above for the one-dimensional diffusion equation, the long channel can be viewed as having a weak “sink” for vapor molecules along its walls; while the evaporation rate will not be as low as for the case of absolutely no condensation on the channel, it should be greatly reduced relative to that of still air over a liquid droplet. This effect will greatly decrease evaporation at the beginning of a long thermal process; as droplets form, the evaporation rate will increase.

This analysis can be applied to the earlier example of long channels partially filled with liquid such that the liquid-vapor interface is much cooler than the reaction chamber. Because the vapor pressure at 60° C. is 2.89 psi (rather than 760 mm Hg or 14.7 psi), c₀ in the above equation will be reduced by a factor of more than 4, leading to an even lower evaporation rate.

A variety of microchip devices may be configured to utilize combinations of applied pressure, reduction in liquid/vapor interfacial area, temperature of the liquid/vapor interface, or hydrophobic coating of chip materials in order to achieve significant reductions in bubble formation and evaporative loss during thermal cycling reactions. For example, the application of pressure alone to a partially-filled channel of sufficiently narrow diameter can lead to negligible evaporation. If a long, sinuous channel 100 μm diameter is filled with 2 μL of liquid (such that the liquid fills a 200 mm length), pressure is applied to prevent bubble formation. The expected evaporation rate extrapolated from empirical observations at 95° C. is 2×10⁻³ μL/sec, as above. For a 40 cycle PCR comprising 5 seconds/cycle at 95° C. plus an initial denaturation of 3 minutes, the total time at 95° C. is 380 sec. The evaporation during these high-temperature steps—which completely dominates evaporation—can then be estimated as <2×10⁻³·380=0.76 μL and the relative loss is 38% of the total liquid; this is sufficient control of evaporation in many cases. As described above, this is a high-end estimate, because it does not take into account diffusional transport of vapor back to the liquid surface; this diffusion and use of hydrophobic coatings can greatly reduce evaporation further, and is in fact the earlier estimate of 2.42×10⁻⁵ μL/sec.

It should be recognized that the cross-sectional area of the channel at the liquid/vapor interface is the dimension governing evaporation, not the overall diameter of channels and chambers. For example, constrictions in the channels leading to a reaction chamber, such as those at capillary valves (typically on the order of 50-100 μm in diameter), can be used, while the channels in which these valves are placed may be of larger diameter for convenience of fluid manipulation.

For example, a microchip substrate according to the present invention may comprise a microchip substrate for performing the method of claim 1, comprising an inlet port, an outlet port, a reaction chamber, a first microchannel fluidly connected to the reaction chamber and the inlet port, and a second microchannel fluidly connected to the reaction chamber and the outlet port, wherein the first and second microchannels each have a diameter less than about half the cross-sectional diameter of the reaction chamber; and the first and second microchannels are each adapted for accepting a pressurized gas.

In another example, a microchip substrate according to the present invention may comprise reaction chamber having a volume of less than about 50 μL and a cross sectional area less than about 0.5 mm². In one embodiment, the reaction chamber comprises a portion of a microchannel having at least one extent of said portion of the microchannel comprising an interface between a liquid in the channel and a pressurized gas. The reaction chamber is not limited by its shape; for example, the reaction chamber may be a straight, U-shaped, ellipsoidal, rectangular, or round channel. In certain embodiments, the reaction chamber has a volume of less than about 25 μL. In other embodiments, the reaction chamber has a volume ranging from about 5 μL to about 50 μL; preferably, the reaction chamber has a volume ranging from about 5 μL to about 25 μL. In another embodiment, and in combination with any volume of the reaction chamber, the cross sectional area of the reaction chamber ranges from about 0.01 mm² (e.g., 10 μm×10 μm) to about 0.5 mm²; preferably, the cross sectional area of the reaction chamber ranges from about 0.1 mm² to about 0.25 mm (e.g., 0.5 mm×0.5 mm).

In general, reaction chambers need not be directly connected via dedicated channels to external ports for application of pressure. Networks of channels and chambers, connected to various input and output ports for fluids, can be simultaneously pressurized to inhibit bubble formation during performance of thermal cycling reactions. For example, in such a device, multiple reaction chambers may be tiled linearly as shown in FIG. 7, which allows for simultaneous thermal cycling of multiple reaction chambers. Tiling chambers in an x-y array is also possible and allows a large number of reaction chambers to be cycled by a single heat source. For example, such arrays can comprise 2, 4, 8, 16, 32, 64, 128, or more reaction chambers in any array (e.g., circular or a geometric grid) which may be simultaneously cycled by a single heat source.

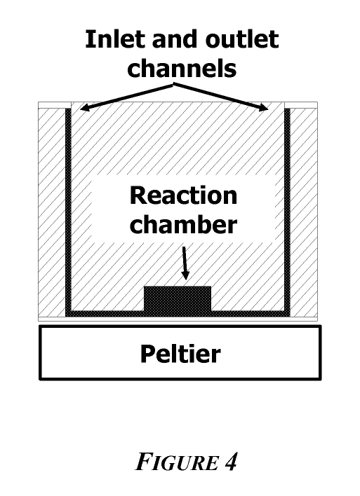

The design elements in chip geometry and thermal cycling are the cross-sectional areas at the liquid/vapor interface—even one which is far inside a network of interconnected channels—and the temperature of those interfaces. For example, a chip may be constructed in which a network of channels is formed in its top surface and are sealed by a thin film or layer. Through-holes penetrating from this network through the body of the chip deliver liquids to cycling chambers on the bottom surface (also sealed by a thin layer or film), as shown in FIG. 4. If thermal cycling occurs via heat transfer on the bottom surface, liquid/vapor interfaces maintained in the small diameter through-holes sufficiently far from the heat transfer surface will be at a lower temperature than the bulk of the liquid, and hence evaporation will be suppressed. Again, the use of hydrophobic materials, and the use of long, small diameter channels leading to the liquid/vapor interfaces, can further reduce evaporation.

For the purposes of this invention, the term “reaction chamber” will be understood to encompass any location within a microchip where a liquid may be isolated, for example, via gas pressurization. Such locations comprise dedicated chambers within the device which are fed by microchannels having cross-sectional diameters smaller than the chamber to which they are in fluid communication as well as a portion of a microchannel itself. For example, any location along a microchannel situated sufficiently near a surface of a microchip to allow for efficient thermal communication with an external heater or cooler (e.g., a Peltier) may be a reaction chamber provided that a liquid sample may be isolated at that location. Isolation may be provided by, for example but not limited to, physical valves, such as hydrogel valves. Positioning of the liquid sample may also be achieved through positive-displacement devices such as syringe pumps, in which the known amount of gas between the syringe plunger (off-chip) and the liquid interface defines the sample position, after which pressurized gas provides isolation and evaporation control. Valveless isolation via the use of passive capillary microvalves may also be employed, as the capillary microvalves define the position of a liquid interface. Positioning of the liquid by application of pressure for a prescribed time may also be used, followed by isolation using applied pressure.

For the purposes of this invention, the term “sample” will be understood to encompass any fluid, solution or mixture, either isolated or detected as a constituent of a more complex mixture, or synthesized from precursor species. In particular, the term “sample” will be understood to encompass any biological species of interest. The term “biological sample” or “biological fluid sample” will be understood to mean any biologically-derived sample, including but not limited to blood, plasma, serum, lymph, saliva, tears, cerebrospinal fluid, urine, sweat, plant and vegetable extracts, semen, and ascites fluid, as well as components thereof at various conditions of purification, particularly nucleic acids. Such nucleic acids may comprise cell lysates as well as purified nucleic acids, each isolated from said biological samples according to methods familiar to those skilled in the art.

For the purposes of this invention, the terms “microfluidics components” and “microfluidic structures” are intended to encompass capillaries, microcapillaries, microchannels, reagent reservoirs, reaction chambers or assay chambers, fluid holding chambers, collection chambers and detection chambers comprising the microchip substrates of the invention, having dimensions for fluid movement of microscale amounts (0.1-100 μL) of fluid.

As used herein, the terms “capillary,” “microcapillary” and “microchannel” will be understood to be interchangeable and to be constructed of either wetting or nonwetting materials where appropriate.

For the purposes of this invention, the terms “entry port” and “fluid input port” will be understood to mean an opening on microchip substrates of the invention comprising a means for applying a fluid to the microchip substrate.

For the purposes of this invention, the terms “exit port” and “fluid outlet port” will be understood to mean a defined volume on microchip substrates of the invention comprising a means for removing a fluid from the microchip substrate.

For the purposes of this invention, the term “pressurized gas” will be understood to encompass gas sources having a pressure greater than about 1.5 atm gauge and can comprise purified nitrogen (e.g., 99+%), compressed air, inert gases, such as argon or helium, and mixtures thereof.

For the purposes of this invention, the term “capillary junction” will be understood to mean a region in a capillary or other flow path in a microfluidics structure of the invention where surface or capillary forces are exploited to retard or promote fluid flow. A capillary junction is provided as a pocket, depression or chamber in a hydrophilic substrate that has a greater depth (vertically within the substrate layer) and/or a greater width (horizontally within the substrate layer) that the fluidics component (such as a microchannel) to which it is fluidly connected. For liquids having a contact angle less than 90° (such as aqueous solutions on microchip substrates made with most plastics, glass and silica), flow is impeded as the channel cross-section increases at the interface of the capillary junction. The force hindering flow is produced by capillary pressure, that is inversely proportional to the cross sectional dimensions of the channel and directly proportional to the surface tension of the liquid, multiplied by the cosine of the contact angle of the fluid in contact with the material comprising the channel.

Capillary junctions can be constructed in at least three ways. In one embodiment, a capillary junction is formed at the junction of two components wherein one or both of the lateral dimensions of one component is larger than the lateral dimension(s) of the other component. As an example, in microfluidics components made from “wetting” or “wettable” materials, such a junction occurs at an enlargement of a capillary. Fluid flow through capillaries is inhibited at such junctions. At junctions of components made from non-wetting or non-wettable materials, on the other hand, a constriction in the fluid path, such as the exit from a chamber or reservoir into a capillary, produces a capillary junction that inhibits flow. In general, it will be understood that capillary junctions are formed when the dimensions of the components change from a small diameter (such as a capillary) to a larger diameter (such as a chamber) in wetting systems, in contrast to non-wettable systems, where capillary junctions form when the dimensions of the components change from a larger diameter (such as a chamber) to a small diameter (such as a capillary).

A second embodiment of a capillary junction is formed using a component having differential surface treatment of a capillary or flow-path. For example, a channel that is hydrophilic (that is, wettable) may be treated to have discrete regions of hydrophobicity (that is, non-wettable). A fluid flowing through such a channel will do so through the hydrophilic areas, while flow will be impeded as the fluid-vapor meniscus impinges upon the hydrophobic zone.

The third embodiment of a capillary junction according to the invention is provided for components having changes in both lateral dimension and surface properties. An example of such a junction is a microchannel opening into a hydrophobic component (microchannel or reservoir) having a larger lateral dimension. Those of ordinary skill will appreciate how capillary junctions according to the invention can be created at the juncture of components having different sizes in their lateral dimensions, different hydrophilic properties, or both.

For the purposes of this invention, the term “capillary action” will be understood to mean fluid flow in the absence of applied external pressure that is due to a partially or completely wettable surface.

For the purposes of this invention, the term “capillary microvalve” will be understood to mean a capillary microchannel comprising a capillary junction whereby fluid flow is impeded and can be motivated by the application of pressure on a fluid. Capillary microvalves will be understood to comprise capillary junctions that can be overcome by increasing the hydrodynamic pressure on the fluid at the junction.

For the purposes of this invention, the term “in fluid communication” or “fluidly connected” is intended to define components that are operably interconnected to allow fluid flow between components.

For the purposes of this invention, the term “air displacement channels” will be understood to include ports in the surface of the microchip substrates that are contiguous with the components (such as microchannels, chambers and reservoirs) on the microchip substrate, and that comprise vents and microchannels that permit displacement of air from components of the microchip substrates by fluid movement.

The microchip substrates of the invention are provided to comprise one or a multiplicity of microsynthetic or microanalytic systems (termed “microfluidics structures” herein). Such microfluidics structures in turn comprise combinations of related components as described in further detail herein that are operably interconnected to allow fluid flow between components upon applied external pressure. For example, a PCR reaction chamber may be in fluid communication with a Sanger sequencing reaction chamber. These components can be microfabricated as described below either integral to the microchip substrates or as modules attached to, placed upon, in contact with or embedded therein. For the purposes of this invention, the term “microfabricated” refers to processes that allow production of these structures on the sub-millimeter scale. These processes include but are not restricted to molding, photolithography, etching, stamping and other means that are familiar to those skilled in the art.

Temperature control elements are provided to control the temperature of the microchip substrate during incubation of a fluid thereupon. The invention therefore provides heating elements, including heat lamps, direct laser heaters, Peltier heat pumps, resistive heaters, ultrasonication heaters and microwave excitation heaters, and cooling elements, including Peltier devices and heat sinks, radiative heat fins and other components to facilitate radiative heat loss. Thermal devices are preferably arrayed to control the temperature of the microchip substrate over a specific area or multiplicity of areas. Preferably, heating and cooling elements comprise or are in thermal contact with the microchip substrate of the invention comprising a thermal regulation layer in or in contact with the microchip substrate surface that is in thermal contact with the microfluidics components, most preferably microchannels as described herein. The temperature of any particular area on the microchip substrates (preferably, the microchannels at any particular thermally regulated area) is monitored by resistive temperature devices (RTD), thermistors, liquid crystal birefringence sensors or by infrared interrogation using IR-specific detectors, and can be regulated by feedback control systems.

In preferred embodiments of the microchip substrates of the invention, the regions of elevated temperatures constructed in the surface of the microchip substrates of the invention comprise a thermal heating element. In preferred embodiments, the thermal heating element is a Peltier device and heat sink, or a resistive heater element or a thermofoil heater, which is an etched-foil heating element enclosed in an electrically insulating plastic (Kapton, obtained from Minco). Alternatively, the microchip substrates of the invention can be used with an external resistive heater, or fluid in the reaction chamber can be heated using localized IR or laser heating. Resistive heater elements comprise in combination an electrically inert substrate capable of being screen printed with a conductive ink and a resistive ink; a conductive ink screen-printed in a pattern; and a resistive ink screen-printed in a pattern over the conductive ink pattern wherein the resistive ink in electrical contact with the conductive ink and wherein an electrical potential applied across the conductive ink causes current to flow across the resistive ink wherein the resistive ink produces heat. Such structures are defined as “electrically-resistive patches” herein. Preferably, the conductive ink is a silver conductive ink such as Dupont 5028, Dupont 5025, Acheson 423SS, Acheson 426SS and Acheson SS24890, and the resistive ink is, for example, Dupont 7082, Dupont 7102, Dupont 7271, Dupont 7278 or Dupont 7285, or a PTC (positive temperature coefficient) ink. In alternative embodiments, the resistive heater element can further comprise a dielectric ink screen-printed over the resistive ink pattern and conductive ink pattern.

Fluid (including reagents, samples and other liquid components) movement is controlled by applied external pressure on the microfluidics components of the substrate. Pressure is applied using, for example, pumping means such as gas cylinders, pumps, and syringe pumps, as well as those disclosed in U.S. Pat. Nos. 5,304,487, 5,498,392, 5,635,358, 5,726,026, 5,928,880 and 6,184,029, the disclosures of each of which are incorporated by reference herein.

The components of the microchip substrates of the invention are in fluidic contract with one another. In preferred embodiments, fluidic contact is provided by microchannels comprising the surface of the microchip substrates of the invention. Microchannel sizes are optimally determined by specific applications and by the amount of and delivery rates of fluids required for each particular embodiment of the microchip substrates and methods of the invention. Microchannel sizes can range from 0.1 μm to a value close to the thickness of the substrate (e.g., about 1 mm); in preferred embodiments, the interior dimension of the microchannel is from 0.5 μm to about 500 μm. Microchannel and reservoir shapes can be trapezoid, circular or other geometric shapes as required. Microchannels preferably are embedded in microchip substrates having a thickness of about 0.1 to 25 mm, wherein the cross-sectional dimension of the microchannels across the thickness dimension of the microchip substrate is less than 1 mm, and can be from 1 to 90 percent of said cross-sectional dimension of the microchip substrate. Sample reservoirs, reagent reservoirs, reaction chambers, collection chambers, detections chambers and sample inlet and outlet ports preferably are embedded in microchip substrates having a thickness of about 0.1 to 2.5 mm, wherein the cross sectional dimension of the microchannels across the thickness dimension of the microchip substrate is from 1 to 75 percent of said cross-sectional dimension of the microchip substrate.

Input and output (entry and exit) ports are components of the microchip substrates of the invention that are used for the introduction or removal of fluid components. Entry ports are provided to allow samples and reagents to be placed on or injected onto the microchip substrate. Exit ports are also provided to allow products to be removed from the microchip substrate. Port shape and design vary according specific applications. For example, sample input ports are designed, inter alia, to allow capillary action to efficiently draw the sample onto the microchip substrate. In addition, ports can be configured to enable automated sample/reagent loading or product removal. Entry and exit ports are most advantageously provided in arrays, whereby multiple samples are applied to the microchip substrate or to effect product removal from the microchip substrate.

In some embodiments of the microchip substrates of the invention, the inlet and outlet ports are adapted to the use of manual pipettors and other means of delivering fluids to the reservoirs of the microchip substrate. In alternative, advantageous embodiments, the microchip substrate is adapted to the use of automated fluid loading devices. One example of such an automated device is a single pipette head located on a robotic arm that moves in a direction along the surface of the microchip substrate.

Also included in air handling systems on the microchip substrate are air displacement channels, whereby the movement of fluids displaces air through channels that connect to the fluid-containing microchannels retrograde to the direction of movement of the fluid, thereby providing a positive pressure to further motivate movement of the fluid.

Microchip substrates of the invention and the microfluidics components comprising such microchip substrates are advantageously provided having a variety of composition and surface coatings appropriate for particular applications. Microchip substrate composition will be a function of structural requirements, manufacturing processes, and reagent compatibility/chemical resistance properties. Specifically, microchip substrates are provided that are made from inorganic crystalline or amorphous materials, e.g. silicon, silica, quartz, inert metals, or from organic materials such as plastics, for example, cyclic olefin polymer (COP) and cyclic olefin co-polymer (COC), poly(methyl methacrylate) (PMMA), acetonitrile-butadiene-styrene (ABS), polycarbonate, polyethylene, polystyrene, polyolefins, polypropylene and metallocene. These may be used with unmodified or modified surfaces as described below. The microchip substrates may also be made from thermoset materials such as polyurethane and poly(dimethyl siloxane) (PDMS). Also provided by the invention are microchip substrates made of composites or combinations of these materials; for example, microchip substrates manufactured of a plastic material having embedded therein an optically transparent glass surface comprising the detection chamber of the microchip substrate. Alternately, microchip substrates composed of layers made from different materials may be made. The surface properties of these materials may be modified for specific applications.

The microchip substrates of the invention can incorporate microfabricated mechanical, optical, and fluidic control components on microchip substrates made from, for example, plastic, silica, quartz, metal or ceramic. These structures are constructed on a sub-millimeter scale by molding, photolithography, etching, stamping or other appropriate means, as described in more detail below. It will also be recognized that microchip substrate comprising a multiplicity of the microfluidics structures are also encompassed by the invention, wherein individual combinations of microfluidics and reservoirs, or such reservoirs shared in common, are provided fluidly connected thereto.

### Microchip Substrate Manufacture and Assembly

Microfluidics structures are provided embedded in microchip substrates of the invention. The microchip substrate can be manufactured and assembled as layers containing separate components that are bonded together. This can be exemplified by a microchip substrate comprising two layers, a reservoir layer and a microfluidics layer. Microchip substrates having additional layers are also within the scope of the invention.

The reservoir layer of the microchip substrates of the invention can be manufactured from a thermoplastic material such as acrylic, polystyrene, polycarbonate, or polyethylene. For such materials, fabrication methods include machining and conventional injection molding. For injection molding, the mold inserts that are used to define the features of the microchip substrate can be created using standard methods of machining, electrical discharge machining, and other means known in the art.

The reservoir layer of the microchip substrates of the invention can be manufactured from a thermoset material or other material that exists in a liquid form until subjected to heat, radiation, or other energy sources. Examples of thermoset materials include poly(dimethyl siloxane) (PDMS), polyurethane, or epoxy. Typically, these materials are obtained from the manufacturer in two parts; the two parts are mixed together in a prescribed ratio, injected into or poured over a mold and subjected to heat to initiate and complete cross-linking of the monomers present in the pre-polymer fluid. The process of rapidly injecting a pre-polymer fluid into a mold and then cross-linking or curing the part is often referred to as reaction injection molding (RIM). The process of pouring a pre-polymer fluid over a mold and then allowing the part to cross-link or cure is often referred to as casting. Mold inserts for RIM or casting can be fabricated using standard methods of machining, electrical discharge machining, and other means known in the art.

The microfluidics layer of the microchip substrate can also be manufactured from a thermoplastic material such as acrylic, polystyrene, polycarbonate, or polyethylene. Because the dimensions of the channels and other microfluidics components may be much smaller than those found in the reservoir layer, typical fabrication methods with these materials may include not only machining and conventional injection molding but also compression/injection molding, and embossing or coining. For injection molding, the mold inserts that are used to define the features of this layer of the microchip substrate can be created using conventional methods such as machining or electrical discharge machining. For mold inserts with features too fine to be created in conventional ways, various microfabrication techniques are used. These include silicon micromachining, in which patterns are created on a silicon wafer substrate through the use of a photoresist and a photomask (Madou, 1997, Fundamentals of Microfabrication, CRC Press: Boca Raton, Fla.). When the silicon wafer is subjected to an etching agent, the photoresist prevents penetration of the agent into the silicon beneath the photoresist, while allowing etching to occur in the exposed areas of the silicon. In this way patterns are etched into the silicon and can be used to create microfabricated plastic parts directly through embossing. In this process, the etched silicon is brought into contact with a flat thermoplastic sheet under high pressure and at a temperature near the glass transition temperature of the plastic. As a result, the pattern is transferred in negative into the plastic.

Etched silicon may also be used to create a metal mold insert through electroplating using, for example, metallic nickel. Silicon etched using any one of a variety of techniques such as anisotropoic or isotropic wet etching or deep reactive ion etching (DRIE) may serve as a basis for a metal mold. A seed layer of nickel is deposited through evaporation on the silicon; once such an electrically-conductive seed layer is formed, conventional electroplating techniques may be used to build a thick nickel layer. Typically, the silicon is then removed (Larsson, 1997, Micro Structure Bull. 1: 3). The insert is then used in conventional injection molding or compression/injection molding.

In addition to silicon micromachining for mold inserts, molds can alternatively be created using photolithography without etching the silicon. Photoresist patterns are created on silicon or other appropriate substrates. Rather than etching the silicon wafer as in silicon micromachining, the photoresist pattern and silicon are metallized through electroplating, thermal vapor deposition, or other means known in the art. The metal relief pattern then serves as a mold for coining, injection molding, or compression/injection molding as described above.

The microfluidic layer of the microchip substrate can also be manufactured using a thermoset material as described above for production of the reservoir layer, wherein the mold pattern for thermosets of the microfluidics layer is prepared as described above. Because reaction-injection molding and casting do not require the high pressures and temperatures of injection molding, a wider variety of mold patterns may be used. In addition to the use of a silicon or metal mold insert, the photoresist pattern as described can also be used as a mold relief itself. While the photoresist would not withstand the high pressures and temperatures of injection molding, the milder conditions of casting or RIM create no significant damage.

The assembly of the microchip substrate involves registration and attachment of the microfluidic layer to the reservoir layer. In order for the microfluidics structures on the microchip substrate to be useful for performing assays as described herein, certain microfluidics pathways in the reservoir layer must be connected to certain microfluidics pathways in the microfluidics layer. Registration of these microfluidics pathways may be accomplished through optical alignment of fiducial marks on the microfluidic and reservoir layers or through mechanical alignment of holes or depressions on the microfluidic layer with pins or raised features on the reservoir layer. The required registration tolerances may be relaxed by designing the microfluidics pathway in the reservoir layer to be much larger than the microfluidics pathway in the microfluidics layer, or vice versa.

Attachment may be accomplished in a number of ways, including conformal sealing, heat sealing or fusion bonding, bonding with a double-sided adhesive tape or heat-sealable film, bonding with a ultraviolet (UV) curable adhesive or a heat-curable glue, chemical bonding or bonding with a solvent.

A requirement for conformal sealing is that one or both of the layers are made of an elastomeric material and that the surfaces to be bonded are free of dust or debris that could limit the physical contact of the two layers. In a preferred assembly approach, an elastomeric microfluidics layer is registered with respect to and then pressed onto a rigid reservoir layer. The elastomeric microfluidics layer may be advantageously made of silicone and the rigid reservoir layer may be advantageously made of acrylic or polycarbonate. Hand pressure allows the layers to adhere through van der Waals forces.

A requirement for heat sealing or fusion bonding is that both the reservoir and microfluidics layers are made of thermoplastic materials and that the sealing occurs at temperatures at or near the glass transition temperatures, in the case of amorphous polymers, or melting temperatures, in the case of semi-crystalline polymers, of both of the layer materials. In a preferred assembly approach, the microfluidics layer is registered with respect to and pressed onto the reservoir layer, this composite disk is then placed between two flat heated blocks and pressure is applied to the composite through the heated blocks. By adjusting the temperature versus time profile at each of the faces of the composite disk and by adjusting the pressure versus time profile that is applied to the composite system, one can determine the time-temperature-pressure profile that allows for bonding of the two layers yet minimizes variation of the features within each of the layers. For example, heating two acrylic disks from room temperature to a temperature just above the glass transition temperature of acrylic at a constant pressure of 250 psi over one hour is a recipe that allows for minimal variation of 250 μm wide fluidic channels. In another assembly approach, the bond surfaces of the microfluidics and reservoir layers are separately heated in a non-contact fashion with radiative lamp and when the bond surfaces have reached their glass transition temperatures the microfluidics layer is registered with respect to and pressed onto the reservoir layer.

A double-sided adhesive tape or heat sealable film may be used to bond the microfluidics and reservoir layers. Before bonding, holes are first cut into the tape (or film) to allow for fluid communication between the two layers, the tape (or film) is registered with respect to and applied onto the reservoir layer, and the microfluidics layer is registered with respect to and applied onto the tape (or film)/reservoir layer composite. In order to bond a heat-sealable film to a surface, it is necessary to raise the temperature of the film to above the glass transition temperature, in the case of an amorphous polymer, or the melting temperature, in the case of a semicrystalline polymer, of the film's adherent polymer material. For bonding with an adhesive tape or a heat-sealable film, an adequate bond can typically be achieved with hand pressure.

A photopolymerizable polymer (for example, a UV-curable glue) or a heat curable polymer may be used to adhere the microfluidics and reservoir layers. In one approach, this glue is applied to one or both of the layers. Application methods include painting, spraying, dip-coating or spin coating. After the application of the glue the layers are assembled and exposed to ultraviolet radiation or heat to allow for the initiation and completion of cross-linking or setting of the glue. In another approach, the microfluidics and reservoir layers are each fabricated with a set of fluid channels that are to be used only for the glue. These channels may, for example, encircle the fluid channels and cuvettes used for the assay. The microfluidics layer is registered with respect to and pressed onto the reservoir layer. The glue is pipetted into the various designated channels and after the glue has filled these channels, the assembled system is exposed to ultraviolet radiation or heat to allow for the crosslinking or setting of the glue.

When polydimethylsiloxane (PDMS) or silicone is first exposed to an oxygen plasma and then pressed onto a similarly treated silicone surface in an ambient environment, the two surfaces adhere. It is thought that the plasma treatment converts the silicone surface to a silanol surface and that the silanol groups are converted to siloxane bonds when the surfaces are brought together (Duffy et al., 1998, Anal. Chem. 70: 4974-4984). This chemical bonding approach is used to adhere the silicone microfluidics and reservoir layer.

A requirement for solvent bonding is that the bond surfaces of both the microfluidics and reservoir layers can be solvated or plasticized with a volatile solvent. For solvent bonding, the bond surfaces are each painted with the appropriate solvating fluid or each exposed to the appropriate solvating vapor and then registered and pressed together. Plasticization allows the polymer molecules to become more mobile and when the surfaces are brought in contact the polymer molecules become entangled; once the solvent has evaporated the polymer molecules are no longer mobile and the molecules remain entangled, thereby allowing for a physical bond between the two surfaces. In another approach, the microfluidics and reservoir layers are each fabricated with a set of fluid channels that are to be used only for the solvent and the layers are bonding much like they are with the UV-curable or heat-curable glue as described above.

Once assembled, the internal surfaces of the microfluidic manifold may be passivated with a solution or 0.01-0.5% polyethylene glycol, bovine serum albumin, or a mixture thereof, or with a parylene coating. Parylene is a vapor-deposited conformal polymer coating that forms a barrier layer on the internal, fluid-contacting surfaces of a microchip substrate following construction. The coating forms an impermeable layer that prevents any exchange of matter between the fluids and materials used to construct the device. The use of a low temperature, vapor deposition method allows the device to be manufactured and then passivated in its final form. This passivation approach can be used to improve the performance of assays. In particular, when an adhesive is used in the construction of the microchip substrate, there is a potential for contamination of the fluids by the adhesive material (or the plastic substrate or cover). Interfering substances leaching from the adhesive, or adsorption and binding of substances by the adhesive, can interfere with chemical or biochemical reactions. This can be more of a problem at elevated temperatures or if solvents, strong acids or bases are required.

### In Vitro Amplification

The invention also provides microchip substrates having microfluidics structures that are able to perform in vitro amplification, and product recovery or analysis. This aspect of the invention is described herein for a single microfluidics structure. However, microchip substrates comprising a multiplicity of these microfluidics structures are provided and are encompassed by the invention, wherein a multiplicity of the microfluidics structures described herein are provided on the microchip substrate.

Generally, thermal cycling is effected in thermal cycling chamber using a variety of thermal cycling protocols and temperature profiles. Examples of such temperature profiles include:
1. Hold the reaction mixture at high temperature (e.g., 95° C.) to denature double stranded DNA
2. Perform a cycle of steps, wherein for n cycles, the following steps are repeated identically n−1 times:
   a) drop the temperature to an annealing temperature (e.g., 45° C.-75° C.), either transiently or for an annealing period to allow annealing of primers to single-stranded DNA;
   b) raise the temperature an extension temperature (e.g., 60° C.-70° C.), either transiently or more preferably with a primer extension period that allows extension of the amplification primers; and
   c) raise the temperature to the denature temperature of the amplified fragment.
      Optionally, the final reaction step comprises dropping the mixture to the annealing temperature and then raising the temperature of the thermal cycling chamber to the extension temperature for a time sufficient to substantially complete the extension reactions on all extended products. Temperature can be cycled using components integral to the microchip substrate, or more preferably the microchip substrate can be adapted for use with an external temperature cycling source. In particular and preferred embodiments, the thermal cycling reaction is performed as set forth herein at pressures greater than atmospheric pressure.

The temperature of the sample is then usually reduced to room temperature or below to stop the reaction.

The following Examples are intended to further illustrate certain preferred embodiments of the invention and are not limiting in nature.

### EXAMPLE 1

Evaporation control using localized heating and filled, narrow channels that terminate at lower temperatures was determined as follows.

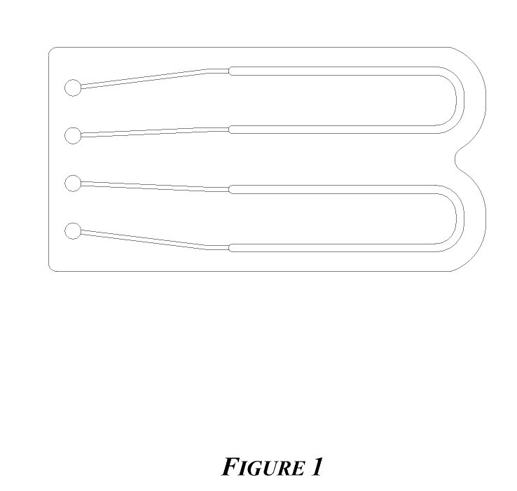

Numerous PCR and Sanger cycling reactions were performed using the device as shown in FIG. 1. These devices contained 5 μL samples and were clamped to a pressure source through O-rings at the ports shown (left). Prior to clamping, the chips were completely filled to the ports with fluid. The chip was then placed on a flat-topped thermal cycler with primarily the narrow loading channels hanging over the edge of the plate, i.e., in air. A pressure of 50 psig N₂ was applied. The chips were then subjected to the following PCR or Sanger sequencing profiles:

PCR profile:

| 1. T = 96° C. | 2 minutes (denaturation) | |
|---|---|---|
| 2. T = 95° C. | 35 | sec |
| 3. T = 66.7° C. | 1 | min 15 s |
| 4. repeat 2-3 | 28 | times |
| 5. 70° C. | 2 | min |

Sanger profile:

| | | |
|---|---|---|
| 1. T = 95° C. | 25 | seconds |
| 2. T = 50° C. | 10 | seconds |
| 3. T = 60° C. | 1 | minute |
| 4. repeat 1-3 | 28 | times |

The channel dimension leading to the large diameter U was 125 μm×250 μm in cross-section. The measured movement of the interfaces from the openings was approximately 3 mm. Thus the volume of liquid loss during the more aggressive Sanger profile was (approximately):

3 mm×2 ×0.125 mm*0.25 mm=0.1875 μL,

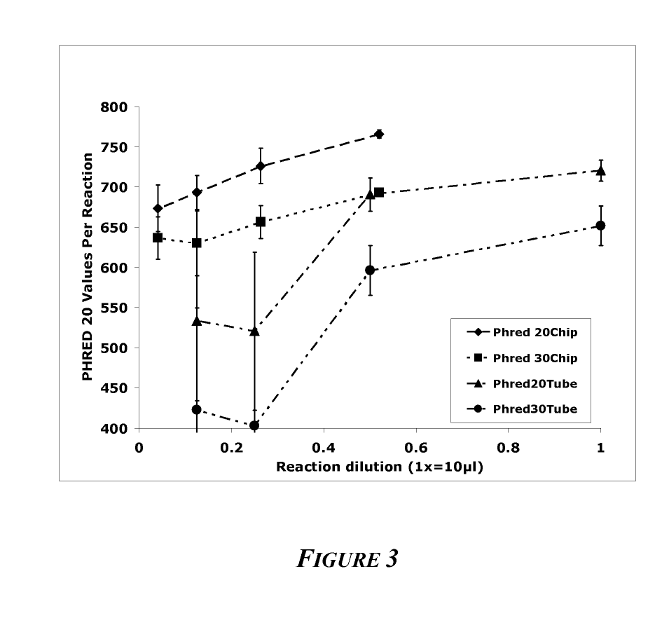

or 3.8%. Sanger sequencing results are shown in FIG. 3. Chemistry is the GE Dyenamic ET Sequencing Kit (GE Healthcare); reaction volume 5 μL and cycled using 50 psi N₂ pressure applied to control evaporation; and sequence analysis on ABI 3730.

This Figure shows that, in terms of PHRED scores, the microchips cycled in this way actually perform better than the tube controls. For comparison, 5 cycles of the same thermal profile for chips that have no seal and no applied pressure were sufficient to cause the entire 5 μL sample to be lost. The performance in chips is superior to that found in tubes, especially for highly-dilute reactions, probably due to the greater temperature uniformity provided in the planar chips relative to PCR tubes.

### EXAMPLE 2

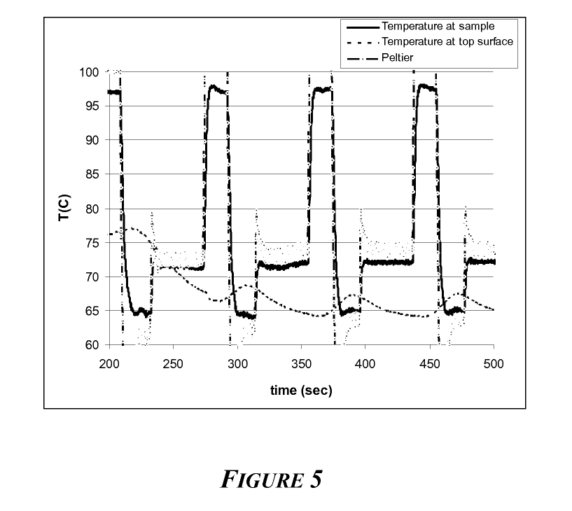

A second example of localized heating and filled, narrow channels permitting easy parallelization is exemplified by a 3-dimensional microchip, in which fluids are added via channels beneath the top surface, as illustrated in FIG. 4. The liquid then passes down to the bottom of the chip, where it fills a chamber. Localized heat is applied at this bottom surface (Peltier), and the filling channels are emptied so that the liquid is confined to the reaction chamber on the bottom, the connecting channels, and the two long, deep holes leading from the top to the bottom. As a result, a large temperature gradient can be provided from the top of the channel to the bottom, leaving two columns of liquid analogous to the fill/empty channels detailed above. When a reaction volume is near a Peltier surface as shown in FIG. 4, fluids are brought from above the surface through narrow channels. While the reaction chamber reaches 95-97° C. required for PCR, the chip top exceeds 75° C. only during the initial denaturation. The steady-state temperature never exceeds 70° C., as shown in FIG. 5. Because the heating/cooling is on the reaction surface of the chip, the top of the chip is much cooler, reducing vapor pressure at the liquid/air interface and thus inhibiting evaporation.

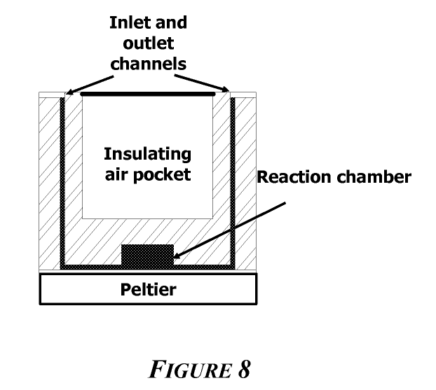

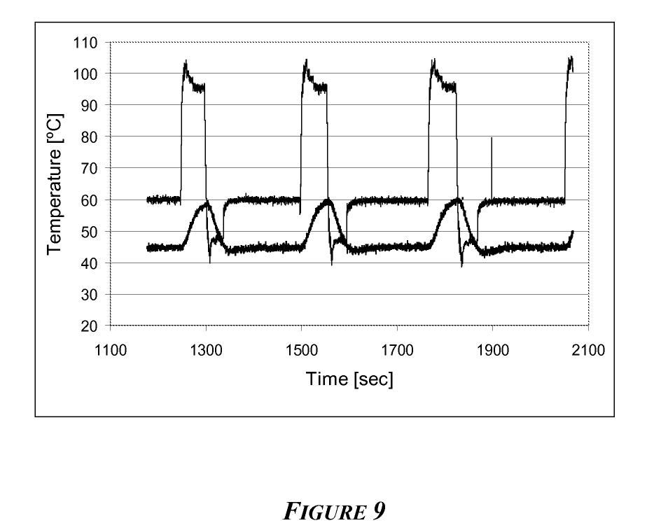

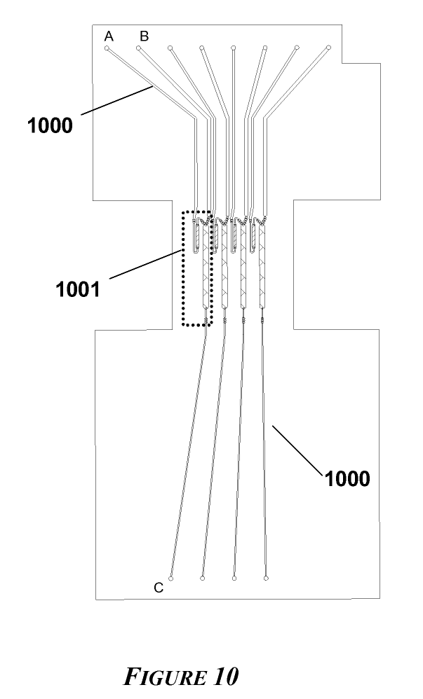

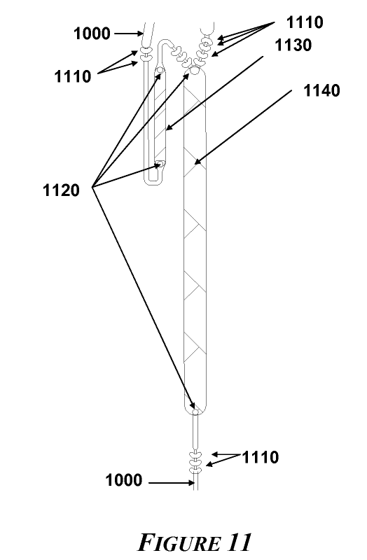

Additionally, an air pocket as provided above the cycling chamber to provide thermal insulation in order to reduce the thermal time-constant in the vicinity of the chamber for rapid thermal cycling, as illustrated in FIG. 8. The insulating air pocket in the chip of FIG. 8 reduces the temperature at the top surface of the chip; see for example FIG. 9, where in the chip of FIG. 8, while the sample temperature reaches ˜100° C. (top curve), the top of the chip never exceeds 60° C. (lower curve).

In one embodiment, the overall thickness of the device is 3.5 mm. The long through-holes have a diameter of 0.34 mm, while the average diameter of the connect channels on the bottom surface is 0.19 mm. The volume of the chamber is 0.39 μL, while that of the channels on the bottom+the through holes is 0.78 μL. As a result, as a simulation of a cycling reaction this would imply that the fraction of the sample at controlled temperature is only 33% and the efficiency of a cycling reaction would be low. As a demonstration of the principle, however, it is effective. Reduction in the cross-channel dimensions of channels to 0.1 mm and a reduction in thickness to 1.5 mm as well as the diameter of the through holes, as well as some reduction in thickness by 50%, should increase the well-controlled reaction volume to 89% of the total volume for a reaction chamber of 0.5 μL size.

This device is placed on a Peltier-controlled thermal cycler and subjected to PCR under applied pressure of 50 psig. Visual inspection at the end of the cycling shows very little bubble formation. By measuring the height of the columns of liquid in the through holes, a loss of reaction mixture, expected to be less than 15% of the reaction mixture volume, is detected.

### EXAMPLE 3

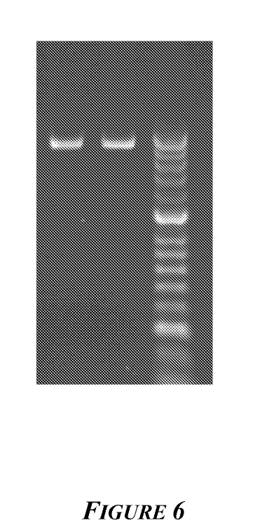

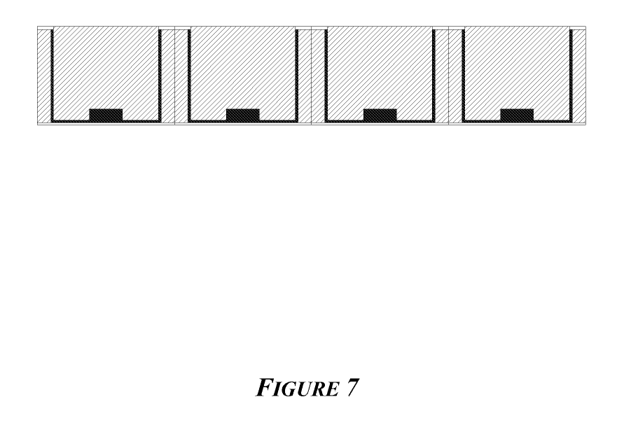

Microchips constructed in the form shown in FIG. 4 were used to perform PCR reactions. A sample containing 1.5 μL of E. coli DH5 transformed with pGEM (˜5×10⁶ cells/μL) was mixed with 1.5 μL PCR reaction mix containing SpeedSTAR™ polymerase (Takara Bio USA) and primer concentration 0.1 μM and introduced into a device as illustrated in FIG. 4. The mixture was cycled on the Peltier surface under applied pressure of 30 psig N₂ with the following parameters: Initial denaturation 96° C. for 3 min (in order to lyse the bacteria and release DNA), then 40 cycles with 96° C. for 20 sec, 65° C. for 15 sec and 72° C. for 45 sec. FIG. 6 shows a gel illustrating a 1.8 kb product retrieved from PCR of the 3 μL sample. The reaction mix showed ˜15% evaporation.

### EXAMPLE 4

In one example of the microchip of the invention, a networked chip can be utilized to perform 4 PCR reactions followed by 4 Sanger reactions. An example of such a microchip is illustrated in FIG. 10 comprising four independent structures within a three layer chip; FIG. 11 shows an expanded view of the boxed area (1001):
1. a thin cover layer (0.375 mm; not shown).

12. a second layer containing the fluidic channels (1000) and capillary microvalves (FIG. 11, 1110); through-holes (FIG. 11, 1120) through the second layer go to the bottom layer
3. a bottom layer in fluid communication with the fluidic channels via the through holes, comprising one or more reaction chambers. For example, in FIG. 11 the bottom layer comprises both a PCR (1130) and Sanger cycling (1140) chamber.

PCR chamber and Sanger chamber are generally in the bottom layer; and all other fluidics in the top of the middle layers (under sealing layer).The microchip of FIGS. 10 and 11 may be used as follows. A 2.5 μL Sample (Bacteria+ PCR mix) is loaded into port A and transported through channels to a through hole leading to the PCR chamber. The liquid emerges through second through hole and is pinned at capillary valve; sufficient volume is added so that liquid also remains in the first through hole, satisfying the “cold interface” condition. Pressure is applied to ports A, B, C. 40 PCR cycles, as described in Example 1, are performed.

8 μL of Sanger reagent is added to port B. Sanger reagent and PCR product are brought together at capillary microvalve on the top of the middle layer. Mixing is by reciprocal motion by applying pressure and vacuum alternately to ports A and B. The Sanger reaction mix is then driven through through hole on distal end of Sanger chamber and is pinned at capillary valve on the top of the middle layer, again satisfying the “cold interface conditions”. Sanger cycling is performed as described in Example 1. The product is retrieved from port C

It should be understood that the foregoing disclosure emphasizes certain specific embodiments of the invention and that all modifications or alternatives equivalent thereto are within the spirit and scope of the invention.

What is claimed is:

1. A method for performing on a microchip an in vitro reaction on a biological sample comprising a nucleic acid, the method comprising, providing a reaction mixture comprising a portion of a biological sample, a polymerase, a buffer, and a primer, to a reaction chamber on a microchip, wherein the biological sample comprises at least one nucleic acid; and subjecting the reaction mixture to a cyclic pattern of temperature changes, wherein the reaction mixture is maintained at a pressure greater than atmospheric pressure during the cyclic pattern of temperature changes, the reaction mixture has at least one liquid-gas interface during the cyclic pattern of temperature changes; and the reaction chamber is maintained at a pressure greater than atmospheric pressure by introducing a pressurized gas.

2. The method according to claim 1, wherein the pressure ranges from about 1.5 atm gauge to about 3 atm gauge.

3. The method according to claim 1, wherein the in vitro reaction is a polymerase chain reaction.

4. The method according to claim 1, wherein the in vitro reaction is a Sanger sequencing reaction.

5. The method according to claim 4, wherein the reaction mixture further comprises dye-labeled ddNTPs or dye labeled primers.

6. The method according to claim 1, wherein the reaction mixture has a volume of less than about 500 μL.

7. The method according to claim 1, wherein the biological sample comprises blood, plasma, serum, lymph, saliva, tears, cerebrospinal fluid, urine, sweat, plant or vegetable extracts, semen, ascites fluid, cell lysates, processed tissues, or nucleic acids isolated from said biological samples.

8. The method according to claim 1, wherein the reaction chamber is a portion of a microchannel isolated by introducing a pressurized gas to the reaction mixture.

9. The method according to claim 1, wherein the at least one liquid-gas interface is maintained at a temperature less than about 80° C. during the cyclic pattern of temperature changes.

10. The method according to claim 1, wherein the reaction chamber is in fluid communication with one or more microchannels.

11. The method of claim 10, wherein the one or more microchannels comprise a hydrophobic surface.

12. The method according to claim 11, wherein the hydrophobic surface is polypropylene or poly(tetrafluoroethylene).

13. The method according to claim 11, wherein at least a portion of the inner surface of the microchannel is treated with a hydrophobic surface coating.

14. The method according to claim 1, wherein the microchip is constructed of an organic material, an inorganic material, a crystalline material or an amorphous material.

15. The method according to claim 14, wherein the microchip comprises silicon, silica, quartz, a ceramic, a metal or a plastic.

16. The method according to claim 15, wherein the microchip is either (i) constructed from a hydrophobic polymer or (ii) further comprises a hydrophobic surface coating.

17. The method according to claim 1, wherein the cyclic pattern of temperature changes are provided by contacting the surface of the microchip proximate to the reaction chamber with a heat source.

18. The method according to claim 17, wherein the heat source is a heat lamp, direct laser heater, Peltier device, resistive heater, ultrasonication heater, or microwave excitation heater.

19. A microchip substrate for performing the method of claim 1, comprising an inlet port, an outlet port, a reaction chamber, a first microchannel fluidly connected to the reaction chamber and the inlet port, and a second microchannel fluidly connected to the reaction chamber and the outlet port, wherein the first and second microchannels each have a diameter less than the cross-sectional diameter of the reaction chamber; and the inlet port and the outlet port are each adapted for accepting a pressurized gas.

20. The microchip substrate according to claim 19 constructed of an organic material, an inorganic material, a crystalline material or an amorphous material.

21. The microchip substrate according to claim 20, comprising silicon, silica, quartz, a ceramic, a metal or a plastic.

22. The microchip substrate according to claim 21, either (i) constructed from a hydrophobic polymer or (ii) further comprising a hydrophobic surface coating.

23. The microchip substrate according to claim 19, wherein the reaction chamber has a volume of less than about 500 μL.

24. The microchip substrate according to claim 19, wherein the reaction chamber is U-shaped.

25. The microchip substrate according to claim 19, comprising a multiplicity of inlet ports, outlet ports, reaction chambers, first microchannels fluidly connected to each of said reaction chambers and inlet ports, and second microchannels fluidly connected to each of said reaction chambers and outlet ports, wherein the first and second microchannels have a diameter less than the cross-sectional diameter of the reaction chamber.

26. The microchip substrate according to claim 19, wherein the first and second microchannels comprise a hydrophobic surface.

27. The microchip substrate according to claim 26, wherein the hydrophobic surface is polypropylene or poly(tetrafluoroethylene).

28. The microchip substrate according to claim 26, wherein at least a portion of the inner surface of the microchannel is treated with a hydrophobic surface coating.

29. A microchip substrate for performing the method of claim 1, comprising a reaction chamber having a volume of less than about 50 μL.

30. The microchip substrate of claim 29, wherein the reaction chamber comprises a portion of a microchannel having at least one extent of said portion of the microchannel comprising an interface between a liquid in the channel and a pressurized gas.

31. The microchip substrate according to claim 29, wherein the reaction chamber is a straight, U-shaped, ellipsoidal, rectangular, or round channel,

32. The microchip substrate according to claim 29, wherein the reaction chamber has a volume of less than about 25 μL.

33. The microchip substrate according to claim 29 constructed of an organic material, an inorganic material, a crystalline material or an amorphous material.

34. The microchip substrate according to claim 33, comprising silicon, silica, quartz, a ceramic, a metal or a plastic.

35. The microchip substrate according to claim 34, either (i) constructed from a hydrophobic polymer or (ii) comprising a hydrophobic surface coating.

36. The microchip substrate according to claim 29, wherein the reaction chamber is in fluid communication with one or more microchannels.

37. The microchip substrate according to claim 36, wherein the one or more microchannels comprise a hydrophobic surface.

38. The microchip substrate according to claim 37, wherein the hydrophobic surface is polypropylene or poly(tetrafluoroethylene).

39. The microchip substrate according to claim 37, wherein at least a portion of the inner surface of the microchannels is treated with a hydrophobic surface coating.

40. A method for performing on a microchip an in vitro reaction on a biological sample comprising a nucleic acid, the method comprising providing a reaction mixture having a liquid-gas interface with a pressurization gas and comprising a portion of a biological sample, a polymerase, a buffer, and a primer to a reaction chamber on a microchip substrate according to claim 19 wherein the biological sample comprises at least one nucleic acid; and subjecting the reaction mixture to a cyclic pattern of temperature changes, having a denaturing temperature, wherein the reaction mixture is maintained at a pressure greater than atmospheric pressure during the cyclic pattern of temperature changes, the reaction mixture has at least one liquid-gas interface during the cyclic pattern of temperature changes; the liquid-gas interface are maintained at a temperature less than the denaturing temperature, and the reaction chamber is pressurized by introducing the pressurized gas.

41. The method according to claim 40, wherein at least a portion of each of the first and second microchannels are maintained at a temperature less than about 80° C.

42. The method according to claim 40, wherein the pressure ranges from about 1.5 atm gauge to about 3 atm gauge.

43. The method according to claim 40, wherein the reaction chamber is in fluid communication with one or more microchannels.

44. The method according to claim 43, wherein and the one or more microchannels comprise a hydrophobic surface.

45. The method according to claim 44, wherein the hydrophobic surface is polypropylene or poly(tetrafluoroethylene).

46. The method according to claim 44, wherein at least a portion of the inner surface of the microchannel is treated with a hydrophobic surface coating.

47. The method according to claim 40, wherein the cyclic pattern of temperature changes are provided by contacting the surface of the microchip proximate to the reaction chamber with a heat source.

48. The method according to claim 47, wherein the heat source is a heat lamp, direct laser heater, Peltier device, resistive heater, ultrasonication heater, or microwave excitation heater.

49. The method according to claim 40, wherein the microchip is constructed of an organic material, an inorganic material, a crystalline material or an amorphous material.

50. The method according to claim 49, wherein the microchip comprised silicon, silica, quartz, a ceramic, a metal or a plastic.

51. The method according to claim 50, wherein the microchip is either (i) constructed from a hydrophobic polymer or (ii) further comprises a hydrophobic surface coating.

52. A method for performing on a microchip an in vitro reaction on a biological sample comprising a nucleic acid, the method comprising providing a reaction mixture having a liquid-gas interface with a pressurization gas and comprising a portion of a biological sample, a polymerase, a buffer, and a primer to a reaction chamber on a microchip substrate according to claim 29 wherein the biological sample comprises at least one nucleic acid; and subjecting the reaction mixture to a cyclic pattern of temperature changes, having a denaturing temperature, wherein the reaction mixture is maintained at a pressure greater than atmospheric pressure during the cyclic pattern of temperature changes, the reaction mixture has at least one liquid-gas interface during the cyclic pattern of temperature changes; the liquid-gas interface is maintained at a temperature less than the denaturing temperature, and the reaction chamber is pressurized by introducing the pressurized gas.

53. The method according to claim 52, wherein the liquid-gas interface is maintained at a temperature less than about 80° C.

54. The method according to claim 52, wherein the pressure ranges from about 1.5 atm gauge to about 3 atm gauge.

55. The method according to claim 52, wherein the reaction chamber is in fluid communication with one or more microchannels.

56. The method according to claim 55, wherein the one or more microchannels comprise a hydrophobic surface.

57. The method according to claim 56, wherein the hydrophobic surface is polypropylene or poly(tetrafluoroethylene).

58. The method according to claim 56, wherein at least a portion of the inner surface of the microchannel is treated with a hydrophobic surface coating.

59. The method according to claim 52, wherein the cyclic pattern of temperature changes are provided by contacting the surface of the microchip proximate to the reaction chamber with a heat source.

60. The method according to claim 59, wherein the heat source is a heat lamp, direct laser heater, Peltier device, resistive heater, ultrasonication heater, or microwave excitation heater.

61. The method according to claim 52, wherein the microchip is constructed of an organic material, an inorganic material, a crystalline material or an amorphous material.

62. The method according to claim 61, wherein the microchip comprised silicon, silica, quartz, a ceramic, a metal or a plastic.

63. The method according to claim 62, wherein the microchip is either (i) constructed from a hydrophobic polymer or (ii) further comprises a hydrophobic surface coating.
